(19) United States
(12) Patent Application Publication
(10) Pub. No.: US 2025/0384013 A1
(43) Pub. Date: Dec. 18, 2025

(54) Deduplicating And Grouping Allergy Events Using Concept Mapping Of Free Text With Large Language Models

(71) Applicant: Cerner Innovation, Inc., Kansas City, MO (US)

(72) Inventors: Suman Pal, Bangalore (IN); Chetan KV, Bangalore (IN); Monica Gaur, Delhi (IN); Rupanjali Chaudhuri, Bangalore (IN); Mayank Sharma, Bhiwani (IN)

(73) Assignee: Cerner Innovation, Inc., Kansas City, MO (US)

(21) Appl. No.: 18/891,267

(22) Filed: Sep. 20, 2024

Related U.S. Application Data
(63) Continuation-in-part of application No. 18/742,412, filed on Jun. 13, 2024.
(60) Provisional application No. 63/692,061, filed on Sep. 7, 2024.

(51) Int. Cl. G06F 16/174; G06F 16/14; G16H 10/60; G16H 40/20

(52) U.S. Cl. CPC G06F 16/1748; G06F 16/14; G16H 40/20; G16H 10/60

(57) ABSTRACT

Techniques for generating recommendations of standard codes for storing in association with allergy free text to facilitate deduplication of patient allergy events are disclosed. Standard codes are alphanumeric identifiers that represent allergy events. Allergy free text is allergy event information in natural language. The system generates vector embeddings for the standard codes by applying a vector embedding function to a set of attributes associated with the standard codes. The system generates a vector embedding for a target unmapped allergy code by applying the vector embedding function to allergy free text of the target unmapped allergy code. The system compares the target vector embedding for the target unmapped allergy code to the vector embeddings computed for each of the standard codes. The system presents recommended standard codes and groupings of similar standard codes to a user for mapping to the allergy free text.

### INCORPORATION BY REFERENCE; DISCLAIMER

Each of the following applications are hereby incorporated by reference: Application No. 63/692,061, filed Sep. 7, 2024; application Ser. No. 18/742,412, filed Jun. 13, 2024. The applicant hereby rescinds any disclaimer of claims scope in the parent application(s) or the prosecution history thereof and advises the USPTO that the claims in the application may be broader than any claim in the parent application(s).

### TECHNICAL FIELD

The present disclosure relates to data deduplication of allergy event records. In particular, the present disclosure relates to deduplicating allergy events associated with free text using natural language processing.

### BACKGROUND

In fostering an open and collaborative healthcare landscape for effective communication among diverse electronic health record (EHR) platforms, progress has been made over the past decade in enabling health data exchange. This progress has resulted in an abundance of information, particularly as relates to a patient's healthcare history, where the patient may have seen multiple healthcare providers belonging to different organizations or healthcare systems. The abundance of information may include redundant or duplicate information.

Prior to prescribing or otherwise administering a medication to a patient, healthcare providers consult with the patient's allergy history. Patient allergy event data includes allergies identified with alphanumeric codes, e.g., standard codes or proprietary codes associated, or allergies identified with allergy free text. Patient allergy event data may be retrieved from numerous sources. Data for individual patient allergies may be received from multiple separate sources, and each source may identify the same patient allergy in a different manner, e.g., a standard code, a different standard code, a proprietary code, and allergy free text, resulting in duplication of patient allergy data.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system in accordance with one or more embodiments;

FIGS. 2A-2C illustrates an example set of operations for deduplicating allergy events using natural language processing in accordance with one or more embodiments;

FIGS. 3A-3C illustrate an example system for deduplication allergy events; and

FIG. 4 illustrates an example of data flow during an example set of operations for presenting a recommendation of candidate standard codes for allergy free text;

FIGS. 5A and 5B illustrate prioritized sorting of recommendations of candidate standardized codes for allergy free text; and

FIG. 6 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. ALLERGY EVENT SYNCHRONIZATION SYSTEM
3. PRESENTING RECOMMENDATIONS OF CANDIDATE STANDARD CODES FOR MAPPING TO UNMAPPED ALLERGY CODES TO FACILITATE ALLERGY EVENT DEDUPLICATION
4. EXAMPLE SYNCHRONIZATION SYSTEM
5. EXAMPLE MAPPING OPERATIONS
6. RECOMMENDATION INTERFACE
7. PRACTICAL APPLICATIONS, ADVANTAGES & IMPROVEMENTS
8. HARDWARE OVERVIEW
9. MISCELLANEOUS; EXTENSIONS

### 1. General Overview

One or more embodiments generate recommendations of standard codes for storing in association with allergy free text to facilitate deduplication of patient allergy events. Standard codes, as referred to herein, are alphanumeric identifiers that represent allergy events. Standard code sets are developed and maintained by organizations and industries involved in healthcare information management, regulation, and standardization. Standard codes facilitate the electronic exchange of allergy-related information, e.g., allergens, between different healthcare systems and organizations. Allergy free text, as referred to herein, is allergy event information in natural language.

Initially, the system generates vector embeddings for the standard codes by applying a vector embedding function to a set of attributes associated with the standard codes. Applying a vector embedding function to the set of attributes includes applying the vector embedding function to text of the set of attributes. The standard codes may comprise a first set of standard codes, e.g., RxNorm, provided by a first entity, e.g., National Library of Medicine (NLM) and a second set of standard codes, e.g., SNOMED CT provided by a second entity, e.g., SNOMED International.

In one or more embodiments, a target unmapped allergy code is represented by allergy free text. The system generates a vector embedding for the target unmapped allergy code by applying the vector embedding function to the allergy free text of the target unmapped allergy code. The system may generate a vector embedding for the allergy free text at least by applying the vector embedding function to an aggregate of the text of the allergy free text. Alternatively, or in addition, the system may apply the vector embedding function to each instance of the allergy free text and combine the resulting vector embeddings to generate the vector embedding for the unmapped allergy code.

In an embodiment, the system compares the target vector embedding for the target unmapped allergy code to the vector embeddings computed for each of the standard codes. Based on a similarity measure between the target vector embedding and the vector embeddings for the unmapped allergy code, the system selects a subset of the standard codes for recommending to the user as a set of candidate standard codes for the target unmapped allergy code. The system presents a group of standard codes, including the candidate standard code and similar codes to a user for selection. When the set of candidate standard codes includes a first candidate standard code from a first set of standard codes from a first entity, and a second candidate standard code from a second set of standard codes from a second entity, the candidate standard codes from the first entity are presented before the candidate standard codes from the second entity. Upon receipt of user input selecting a particular standard code, of the set of candidate standard codes, the system stores an association, or mapping, between the allergy free text and the particular standard code.

In one or more embodiments, the system identifies a second allergy event associated with a second standard code as being the same as the first standard code. The system removes one of the first or second allergy event from the patient allergy data as being duplicative of the other of the first or second allergy event.

One or more embodiments described in this Specification and/or recited in the claims may not be included in this General Overview section.

### 2. Allergy Synchronization System

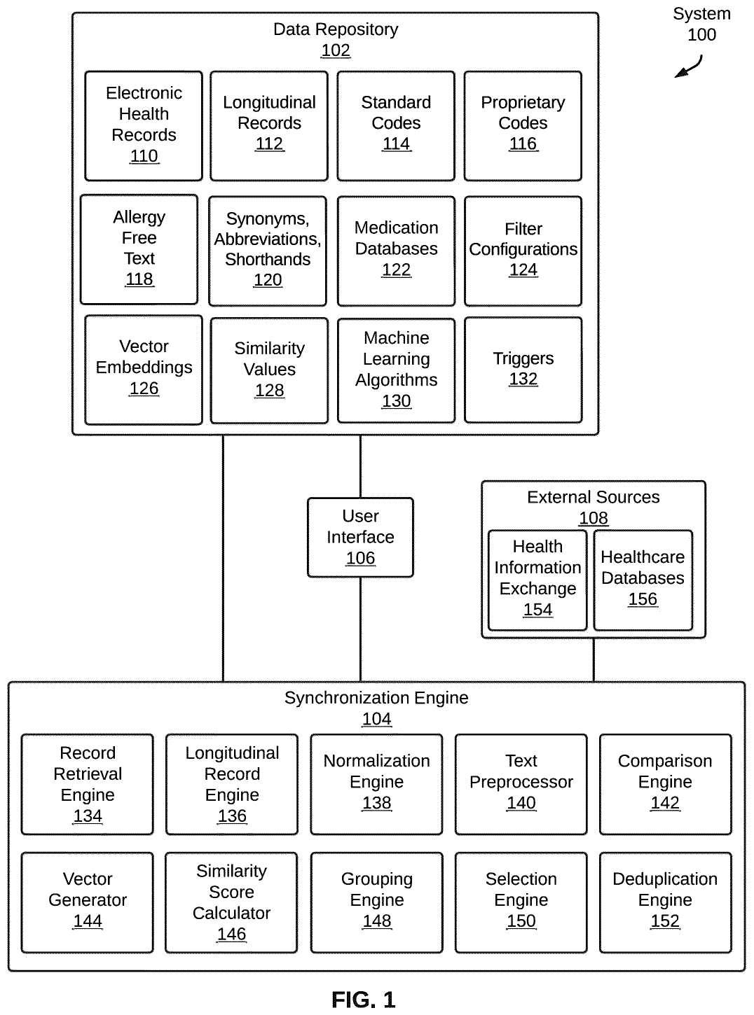

FIG. 1 illustrates a system 100 in accordance with one or more embodiments. As illustrated in FIG. 1, system 100 includes a data repository 102, a synchronization engine 104, a user interface 106, and external sources 108. In one or more embodiments, the system 100 may include more or fewer components than the components illustrated in FIG. 1. The components illustrated in FIG. 1 may be local to or remote from each other. The components illustrated in FIG. 1 may be implemented in software and/or hardware. Each component may be distributed over multiple applications and/or machines. Multiple components may be combined into one application and/or machine. Operations described with respect to one component may instead be performed by another component.

In one or more embodiments, a data repository 102 is any type of storage unit and/or device (e.g., a file system, database, collection of tables, or any other storage mechanism) for storing data. Further, a data repository 102 may include multiple different storage units and/or devices. The multiple different storage units and/or devices may or may not be of the same type or located at the same physical site. Further, a data repository 102 may be implemented or executed on the same computing system as the synchronization engine 104 and the user interface 106. Additionally, or alternatively, a data repository 102 may be implemented or executed on a computing system separate from the synchronization engine 104 and the user interface 106. The data repository 102 may be communicatively coupled to the synchronization engine 104 and the user interface 106 via a direct connection or via a network.

Information describing operations for deduplicating patient allergy events using nature language processing may be implemented across any of components within the system 100. However, this information is illustrated within the data repository 102 for purposes of clarity and explanation.

In embodiments, the data repository 102 is populated with information from a variety of sources and/or systems. The data repository 102 may include electronic healthcare records (EHRs) 110, longitudinal records 112, standard codes 114, proprietary codes 116, allergy free text 118, synonyms, abbreviations, and shorthands 120, a medication database 122, filter configurations 124, vector embeddings 126, similarity values 128, machine learning algorithms 130, and triggers 132. Any of this information may be stored in a structured format (e.g., a table).

In one or more embodiments, EHRs 110 are digital versions of healthcare records. EHRs 110 comprise medical history, diagnoses, medications, treatment plans, immunization dates, allergies, radiology images, laboratory test results, and/or other patient information. EHRs 110 may be from the same or different systems and/or providers. Examples of EHR providers include Cerner Millenium and Epic.

In embodiments, EHRs 110 are populated with codes associated with patient allergy events. Patient allergy events include instances of a patient experience an adverse reaction to a medication, food, or other substance. The codes associated with the patient allergy events may include standard codes 114, proprietary codes 116, allergy free text 118, and/or a combination of codes and text. For example, a first EHR may identify patient allergy events with proprietary codes and a second EHR may identify patient allergy events with allergy free text. Similarly, a first EHR may identify a first allergy event with a first standard code and a second EHR may identify the same first allergy event with a second standard code.

In one or more embodiments, longitudinal records 112 are comprehensive and cumulative records that document a patient's health information over time. Unlike traditional medical records, which may only capture a snapshot of a patient's health status at a specific point in time, longitudinal records provide a longitudinal view of the patient's health history, diagnoses, treatments, medications, allergies, procedures, and outcomes across multiple encounters and care settings.

In embodiments, longitudinal records 112 offer continuity, comprehensiveness, timeliness, accessibility, and interoperability. Longitudinal records 112 span the entire continuum of care, capturing information from various healthcare encounters, including primary care visits, specialist consultations, hospitalizations, emergency department visits, diagnostic tests, and procedures. Longitudinal records 112 encompass a wide range of health information, including medical history, social history, family history, allergies, medications, immunizations, laboratory results, imaging studies, progress notes, care plans, and outcomes.

In some embodiments, longitudinal records 112 are updated in real-time or near-real-time as new health information becomes available. Longitudinal records 112 are accessible to authorized healthcare providers and patients across different care settings and healthcare organizations. EHR systems, health information exchanges (HIEs), and patient portals facilitate the sharing and exchange of longitudinal health information. Longitudinal records 112 support interoperability between different healthcare systems and applications, allowing seamless exchange and integration of health information across disparate platforms. Standards-based data exchange protocols, terminologies, and coding systems promote interoperability and data exchange among healthcare stakeholders.

In one or more embodiments, standard codes 114 are alphanumeric identifiers used to represent medications or allergies in healthcare settings. Standard code sets are developed and maintained by organizations and industries involved in healthcare information management, regulation, and standardization. Standard codes 114 facilitate the electronic exchange of medication and allergy related information between different healthcare systems and organizations.

Some widely used standard code systems include, Anatomical Therapeutic Chemical Classification System (ATC), LOINC (Logical Observation Identifiers Names and Codes), and Health Level Seven (HL7) Standard Codes. The NDC is a unique 10-digit, 3-segment numeric identifier assigned to medications in the United States by the Food and Drug Administration (FDA). The NDC identifies the manufacturer or distributor, the product, and the package size or dosage form of the medication. The ATC system is an international classification system developed by the World Health Organization (WHO) for the classification of drugs based on their therapeutic and pharmacological properties. The ATC system uses alphanumeric codes to categorize medications into different anatomical groups, therapeutic groups, and chemical subgroups. While primarily used for laboratory tests and clinical observations, LOINC also includes codes for clinical drug names and medication-related concepts to support interoperability in electronic health records (EHRs) and health information exchanges (HIEs). HL7 includes standard code systems for representing medications, such as those used in HL7 Version 2 and HL7 Version 3 messaging standards, to facilitate the exchange of medication-related information between healthcare systems and applications.

In one or more embodiments, the standard codes 114 are RxNorm. RxNorm is a standardized nomenclature for clinical drugs developed by the NLM. RxNorm provides normalized names and codes for clinical drugs, including brand names, generic names, and ingredients, to facilitate electronic prescribing and medication reconciliation. RxNorm uses Term Types (TTYs) to indicate generic and branded drug names at different levels of specificity. TTYs are semantic tags that describe the type of information the concept conveys.

TTYs include route, dosage, ingredient (IN), precise ingredient (PIN), multiple ingredients (MIN), semantic clinical drug (SCD), semantic branded drug (SBD), brand name pack (BPCK), and generic pack (GPCK). Route refers to the path or method by which a medication is administered or delivered into the body. Route specifies how a drug is introduced to the patient's system, indicating whether the drug is taken orally, injected, applied topically, inhaled, or administered through other routes. Common examples of medication routes include oral (by mouth), intravenous (IV), intramuscular (IM), subcutaneous (SC), topical (applied to the skin), and inhalation. Dosage refers to the specific amount or quantity of a drug prescribed for an individual patient during a given period. It is a crucial component of a medical prescription and is often expressed in terms of units of the drug (such as milligrams or micrograms) per unit of the patient (such as kilograms or pounds) and the frequency of administration (such as once daily or twice a day). Ingredient (IN) is a compound or moiety that gives the drug its distinctive clinical properties. Ingredients generally use the United States Adopted Name (USAN). Example: Fluoxetine. Precise Ingredient (PIN) is a specified form of the ingredient that may or may not be clinically active. The most precise ingredients are salt or isomer forms. Example: Fluoxetine Hydrochloride Multiple Ingredients (MIN) are two or more ingredients appearing together in a single drug preparation, created from Semantic Clinical Drug Form (SCDF). Clinical Drug (SCD): Ingredient+Strength+Dose Form. Example: Fluoxetine 4 MG/ML Oral Solution. Semantic Branded Drug (SBD): Ingredient+Strength+Dose Form+Brand Name. Example: Fluoxetine 4 MG/ML Oral Solution [Prozac]. Brand Name Pack (BPCK) is {#(Ingredient Strength Dose Form)/#(Ingredient Strength Dose Form)} Pack [Brand Name]. Example: {12 (Ethinyl Estradiol 0.035 MG/Norethindrone 0.5 MG Oral Tablet)/9 (Ethinyl Estradiol 0.035 MG/Norethindrone 1 MG Oral Tablet)/7 (Inert Ingredients 1 MG Oral Tablet)} Pack [Leena 28 Day]. Generic Pack (GPCK) is {#(Ingredient+Strength+Dose Form)/#(Ingredient+Strength+Dose Form)} Pack. Example: {11 (varenicline 0.5 MG Oral Tablet)/42 (varenicline 1 MG Oral Tablet)} Pack.

In one or more embodiments, the standard codes 114 are Systematized Nomenclature of Medicine Clinical Terms (SNOMED CT) provided by SNOMED International. SNOMED CT is a comprehensive clinical terminology system used internationally to represent clinical concepts in healthcare. SNOMED CT includes codes for medications, as well as other clinical concepts, e.g., allergies, procedures, and observations, to support interoperability and semantic interoperability in healthcare information systems. SNOMED CT provides specific codes to represent various types of allergic reactions, allergens, and conditions related to allergies. SNOMED CT codes for allergic reactions represent different types of allergic responses, such as, mild allergic reaction, severe allergic reaction, anaphylaxis. SNOMED CT codes for allergen substances represent specific allergens, such as, peanut allergen—91935009, pollen allergen—418689008, and penicillin allergen—294614009. SNOMED CT codes for allergic conditions represent various allergy-related conditions, such as, allergic rhinitis—61582004, food allergy—91934004, drug allergy—419511003. SNOMED CT codes for history of allergies capture a patient's history of allergies, such as, history of allergy to substance—420134006, and family history of allergy—160301000119100.

SNOMED CT organizes clinical terms into various categories (hierarchies), or semantic tags, to provide a structured and standardized way to document healthcare information. The categories include Substance, Product, Finding, Medicinal Product, Physical Object, Organism, Situation, and Qualifier Value. Substance includes chemical entities and materials, which can be used in or interact with the human body. Substance can be allergens, active pharmaceutical ingredients, chemicals, or other materials. Examples of Substance include Penicillin: SNOMED CT Code 373270004, Latex: SNOMED CT Code 726354002, and Pollen: SNOMED CT Code 406455002. Product covers manufactured products that may include one or more substances, particularly in the context of healthcare. Product can be medical devices, pharmaceutical products, or other therapeutic goods. Examples of Product include Epinephrine auto-injector: SNOMED CT Code 425293005, Peanut-containing food: SNOMED CT Code 761021000000108, and Insulin injection product: SNOMED CT Code 82271004. Finding includes clinical observations, symptoms, diagnoses, and other findings relevant to patient care. Finding is used to document the presence or absence of clinical phenomena. Examples of findings include Allergic rhinitis: SNOMED CT Code 61582004, Urticaria (hives): SNOMED CT Code 247472004, and Anaphylaxis: SNOMED CT Code 39579001. Medicinal Product is a subcategory under “Product” but specifically refers to regulated products intended for therapeutic use. Medicinal products can be drugs, vaccines, or any formulation used for treating or preventing disease. Examples of Medicinal Product include Amoxicillin: SNOMED CT Code 372687004, Aspirin: SNOMED CT Code 119727009, and Influenza vaccine: SNOMED CT Code 70685002. Physical Object includes tangible objects used in healthcare, such as medical devices, instruments, and other physical entities. Examples of Physical Object include Syringe: SNOMED CT Code 264180000, Inhaler: SNOMED CT Code 705019005, and Stethoscope: SNOMED CT Code 7771000. Organism includes living entities such as bacteria, viruses, fungi, plants, and animals, which can be involved in or cause clinical conditions. Examples of Organism include Staphylococcus aureus: SNOMED CT Code 3092008, Dust mite: SNOMED CT Code 415212007, and Candida albicans: SNOMED CT Code 406516002. Situation (with Explicit Context) includes situations or contexts that provide additional information about a clinical finding, procedure, or event. It often involves historical, familial, or social context. Examples of Situation include History of allergy to penicillin: SNOMED CT Code 419076005, Family history of asthma: SNOMED CT Code 312850006, and Exposure to allergen: SNOMED CT Code 429504002. Qualifier Value provides additional detail or modifiers to other SNOMED CT concepts. Qualifier Value are not standalone concepts but are used to refine or specify the meaning of other concepts. Examples of Qualifier Value include Severe: SNOMED CT Code 24484000, Mild: SNOMED CT Code 255604002, and Bilateral: SNOMED CT Code 51440002.

In one or more embodiments, the standard codes 114 are a combination of RxNorm and SNOMED CT. SNOMED CT codes may be preferred over RxNorm codes when the allergy data is associated with food and/or substances. RxNorm may be preferred over RxNorm when the allergy data is associated with medication.

In one or more embodiments, proprietary codes 116 are identifiers specific to particular healthcare organizations, pharmacy chain, or electronic health record (EHR) system. Unlike standard codes 114, which follow universally accepted standards and are designed for interoperability between different systems, proprietary codes 116 are internal to a specific organization's database or system. The proprietary codes 116 may be used for various purposes within the organization, including inventory management, billing, internal communication, and data analytics. Proprietary codes 116 often provide additional information or functionality tailored to the organization's specific needs and workflows.

In one or more embodiments, allergy free text 118 refers to patient allergy events identified using natural language or plain text. In this manner, healthcare providers document allergy events using plain text, rather than selecting allergies from a predefined list or database. In healthcare settings, health care providers may have the option to enter allergies using free text fields in electronic health record (EHR) systems. Allergy free-text may include medication names, substances, abbreviations, acronyms, or shorthand notations that are not standard or universally recognized, and notes, comments, or annotations associated with allergy events that provide additional context or information but are not coded or standardized.

In embodiments, allergy free text 118 provides flexibility and allows healthcare providers to document medications, foods, and substances in a format preferred by the healthcare provider. The use of allergy free text 118 introduces challenges related to accuracy, standardization, and interoperability. Allergy free text 118 is prone to errors, such as misspellings, abbreviations, or incomplete information. Errors can lead to misinterpretation by other healthcare providers.

In some embodiments, the synonyms, abbreviations, and shorthands 120 are included in a table that provides synonyms, abbreviations, and/or shorthands that may or may not be specific to a consumer and corresponding expansions for the respective synonym, abbreviation or shorthand. For example, “qd” may refer to once a day, “bid” may refer to twice a day, “ac” may refer to before meals, “po” may refer to orally, “q4h” may refer to every four hours, and “qod” may refer to every other da. Other abbreviations may relate to specific medications or compounds, e.g., “ZnSO4” for zinc sulfate and “Acetamin” for acetaminophen.

In one or more embodiments, medication database 122 is a structured collection of data containing comprehensive information about medications, e.g., Multum, Lexicomp, Micromedex, and allergies. The medication database 122 serves as a central repository of medication-related data that can be accessed, queried, and utilized by healthcare professionals, researchers, and software applications for various purposes, such as prescribing, dispensing, administration, monitoring, and research. The medication database 122 may include the standard codes 114, proprietary codes 116, allergy free text 118, synonyms, abbreviations, shorthands 120, code mappings, and allergy code groupings.

In one or more embodiments, medication databases 122 include medication information, drug interactions, and clinical guidelines. Medication information is detailed information about medications, including generic and brand names, dosages and strengths, routes of administration, formulations and dosage forms, indications and uses, contraindications and warnings, and side effects and adverse reactions. Drug Interactions are information about potential interactions between medications, including drug-drug interactions, drug-food interactions, drug-allergy interactions, pharmacokinetic interactions, pharmacodynamic interactions. Clinical Guidelines are recommendations and guidelines for safe and effective medication use, including dosage recommendations, administration guidelines, monitoring parameters, special populations considerations (e.g., pediatric, geriatric, pregnancy), and treatment algorithms and protocols.

In one or more embodiments, medication databases 122 includes formulary management, coding and classification, and references and citations. Formulary management is information about medications included in healthcare organization formularies, including preferred drug lists, drug utilization reviews, therapeutic interchange programs, medication cost and reimbursement information. Regulatory information is compliance and regulatory data related to medications, including: FDA approvals and labeling information, drug scheduling and controlled substance classifications, black box warnings and safety alerts, post-marketing surveillance data. Coding and classification is standardized coding systems and classification schemes for medications and allergies, such as NDC, RxNorm, ATC, SNOMED CT. This may include mappings for mapping a first set of standard codes to a second set of standard codes, or mapping standard codes 114 to proprietary codes 116, and/or mapping standard codes 114 to allergy free text 118. Mappings may also include remappings, i.e., between inactive codes and active codes. Coding and classification may also include groupings of like or similar standard codes 114, e.g., brand name and generic medications. References and citations are sources of medication information, including pharmacology textbooks and reference books, clinical practice guidelines, research articles and scientific literature, drug manufacturer package inserts.

In one or more embodiments, the filter configurations 124 determine how a normalization engine 138 of the synchronization engine 104 filters and sorts the patient allergy events. The patient allergy data may be sorted by code into like or similar groups or buckets. Patient allergy events identified with standard codes may be separated from patient allergy events identified with allergy free text. Patient allergy events not associated with a known code or allergy free text may be removed from the patient allergy data.

In one or more embodiments, the vector embeddings 126 in the data repository 102 include text that has been converted to a numeric format. The vector embeddings 126 are representations of individual words for text analysis, typically in the form of a real-valued vector. The vector embeddings 126 may represent individual text items or may represent an aggregation of text items. As will be described in further detail below with respect to synchronization engine 104, the vector embeddings 126 may be formed using various word embedding techniques. The vector embeddings 126 represent standard codes and allergy free text.

In some embodiments, the similarity values or metrics 128 in the data repository 102 provide an indication of the similarity between the vector embeddings 126 for standard codes and allergy free text. The higher the similarity values 128 (for example, the closer to 1.0, depending on the scale), the greater a semantic match between the vector embeddings 126 of a standard code and an allergy free text. The similarity values 128 may be assigned a ranking category. For example, a similarity value less than 0.90 may be categorized as “low”; a similarity value equal to or greater than 0.90 and less than 0.98 may be categorized as “medium”; and a similarity value greater than or equal to 0.98 may be categorized as “high.” The similarity values 112 may be weighted to reflect the relevance of the type of data used to calculate the vector embeddings 126.

In one or more embodiments, a machine learning algorithm 130 is an algorithm that can be iterated to train a target model f that best maps a set of input variables to an output variable. In particular, a machine learning algorithm 130 is configured to generate and/or train a semantic similarity model or a deduplication model.

A machine learning algorithm is an algorithm that can be iterated to train a target model f that best maps a set of input variables to an output variable, using a set of training data. The training data includes datasets and associated labels. The datasets are associated with input variables for the target model f. The associated labels are associated with the output variable of the target model f. The training data may be updated based on, for example, feedback on the predictions by the target model f and accuracy of the current target model f. Updated training data is fed back into the machine learning algorithm, which in turn updates the target model f.

A machine learning algorithm 130 generates a target model f such that the target model f best fits the datasets of training data to the labels of the training data. Additionally, or alternatively, a machine learning algorithm 130 generates a target model f such that when the target model f is applied to the datasets of the training data, a maximum number of results determined by the target model f matches the labels of the training data. Different target models may be generated based on different machine learning algorithms and/or different sets of training data.

A machine learning algorithm may include supervised components and/or unsupervised components. Various types of algorithms may be used, such as linear regression, logistic regression, linear discriminant analysis, classification and regression trees, naïve Bayes, k-nearest neighbors, learning vector quantization, support vector machine, bagging and random forest, boosting, backpropagation, and/or clustering.

In one or more embodiments, triggers 132 are actions that initiate a synchronization process between different systems or devices without requiring manual intervention. Triggers 132 automate the synchronization workflow, ensuring that data remains consistent and up to date across all synchronized endpoints.

In one or more embodiments, triggers 132 include scheduled synchronization, event-based triggers, threshold-based triggers, system startup or shutdown, manual override, dependency-based triggers, external events or conditions. Synchronization can be scheduled to occur at specific intervals, such as hourly, daily, or weekly. Synchronization can be triggered by specific events or actions, such as the creation, modification, or deletion of data records, scheduling of future appointments, admittance, discharge and transfer of a patient. For example, when a new record is added to one system, an event-based trigger can initiate synchronization to propagate the new record to other synchronized systems in real-time. Synchronization can be triggered based on predefined thresholds or conditions. For example, synchronization may be triggered when the number of pending changes exceeds a certain threshold or when a specific data condition is met. Synchronization can be triggered automatically when a system or application starts up or shuts down.

While automatic triggers handle most synchronization scenarios, manual triggers can also be implemented to allow users to initiate synchronization manually when needed. Manual override triggers provide flexibility for users to synchronize data on-demand, especially in situations where immediate synchronization is required. Synchronization can be triggered based on dependencies between data elements or systems. For example, if changes to a particular data element depend on changes to another related data element, synchronization can be triggered automatically when the dependent data element is modified. Synchronization can be triggered by external events or conditions detected by external systems or sensors. For example, synchronization may be triggered in response to changes in environmental conditions, healthcare trends, or other external factors that affect the data being synchronized.

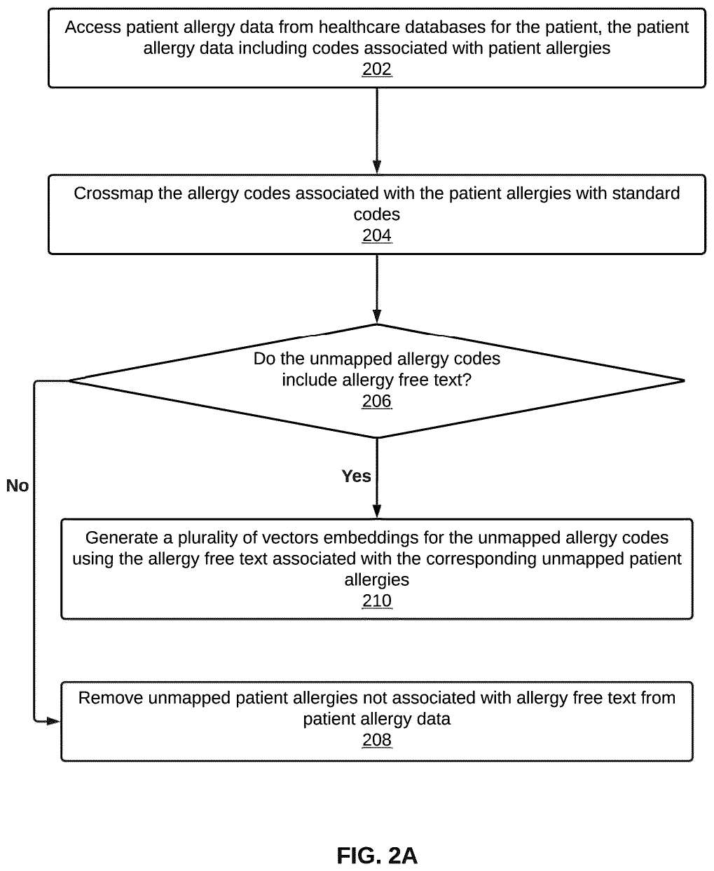

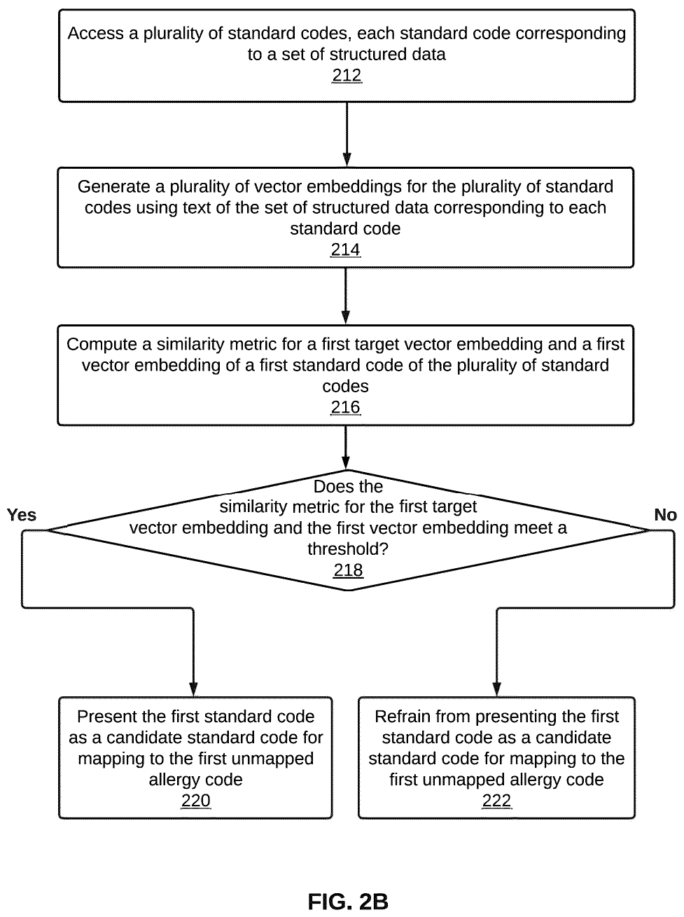

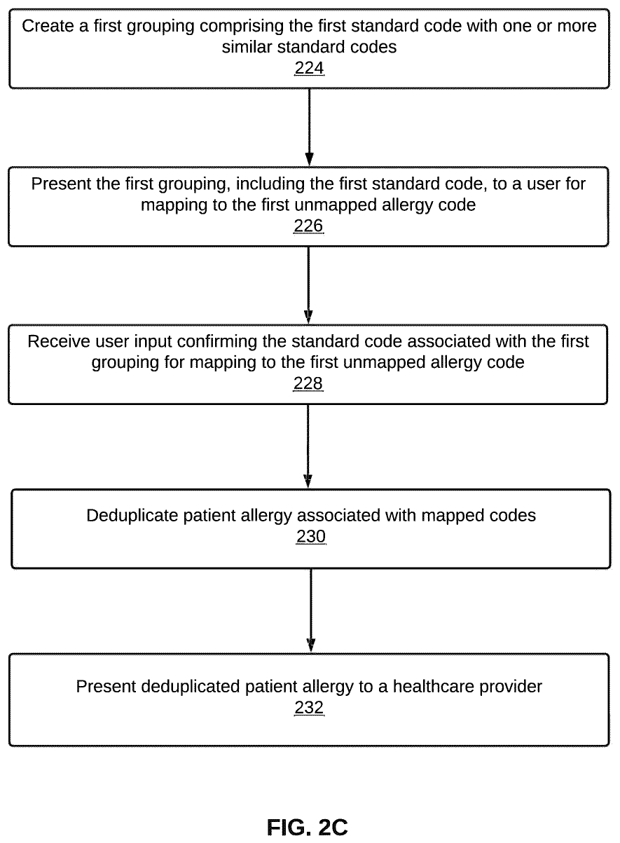

In one or more embodiments, synchronization engine 104 refers to hardware and/or software configured to perform operations described herein for deduplicating patient allergy records using natural language processing to map allergy free text to standard codes. Examples of operations for mapping allergy free text to standard codes to assist in deduplicating patient allergy records are described below with reference to FIGS. 2A-2C.

In an embodiment, synchronization engine 104 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

In one or more embodiments, the synchronization engine 104 includes a record retrieval engine 134, a longitudinal record engine 136, a normalization engine 138, a text preprocessor 140, a comparison engine 142, a vector generator 144, a similarity score calculator 146, a grouping engine 148, a selection engine 150, and a deduplication engine 152.

In one or more embodiments, the record retrieval engine 134 is a software component or system that facilitates the retrieval of records or data from one or more databases or data repositories based on specified criteria or queries. The record retrieval engine 134 processes user queries or search criteria to identify relevant records or data within the database. Users can specify criteria such as keywords, filters, or conditions to narrow down the search and retrieve specific records. The record retrieval engine 134 aggregates data from various sources and healthcare encounters, including EHRs, hospital information systems (HIS), laboratory information systems (LIS), radiology information systems (RIS), pharmacy systems, and other clinical data repositories. Alternatively, data retrieval and aggregation are provided by the record retrieval engine 134. The record retrieval engine 134 consolidates disparate data sources to create a unified and comprehensive view of the patient's health information. The record retrieval engine 134 integrates with different healthcare systems and applications using interoperability standards and interfaces, such as HL7, FHIR, or proprietary Application Programming Interfaces (APIs). This allows the record retrieval engine 134 to retrieve and harmonize data from multiple sources, regardless of vendor or system type.

In one or more embodiments, the longitudinal record engine 136, also known as a longitudinal health record system or longitudinal patient record system, is a software platform or component designed to organize and present comprehensive health information about an individual patient over time. The longitudinal record engine 136 provides a longitudinal view of the patient's health history, medical encounters, treatments, medications, test results, and other relevant clinical data across different healthcare settings and encounters.

In one or more embodiments, the normalization engine 138 is a software component or system that standardizes and normalizes data from heterogeneous sources, ensuring consistency in terminology, coding, and formatting. The normalization engine 138 maps and translates data elements to standardized vocabularies, coding systems (e.g., SNOMED CT, LOINC, RxNorm), and data models to promote interoperability and semantic consistency. The normalization engine 138 may also perform filtering of the patient allergy event data based on the filter configurations 124.

In some embodiments, the text preprocessor 140 is a software component or system that performs functions such as converting the text into lowercase, removing white spaces, prefix removal, punctuation removal, and/or retaining numeric tokens. Text is converted to lowercase to provide uniformity to the text. Prefix removal includes removing prefixes such as “z,” “zz,” “zzz.” Punctuation removal is performed to remove any non-alphanumeric characters. In prior art mapping engines, numeric tokens are typically removed during text preprocessing. Removal of numeric tokens may eliminate a distinguishing feature of an allergy free text. For example, “lamictal 25 mg tablet” and “lamictal 50 mg tablet” are differentiated using a numeric token. By retaining numeric tokens, misclassifications are more readily avoided.

In embodiments, text preprocessing may further include handling special characters, removing unwanted text, and custom preprocessing. Handling special characters includes addressing symbols and special characters. For example, text line “D-Dimer” requires special attention. Replacing the “-” with a blank space creates two different tokens, namely “D” and “Dimer.” As such, using traditional text preprocessing, the entire context of “D-Dimer” is lost. By addressing special characters, the context of the terms is maintained. Custom preprocessing includes attending to consumer specific text such as synonyms, abbreviations, and shorthands. The custom preprocessing may consult the synonyms, abbreviations, and shorthands 120 stored in the data repository 102 to provide expansions for various consumer specific synonyms, abbreviations, and shorthands.

In one or more embodiments, the comparison engine 142 is a software component or system designed to analyze and match codes comprising allergy free text against previously mapped allergy free text. The comparison engine 142 uses lexical search to identify allergy free text matching that has already been mapped to a standard code 114.

In some embodiments, the vector generator 144 includes software and/or hardware for performing one or more vector embedding functions. Vector embedding functions are mathematical functions that map objects, such as words, sentences, or other data points, into vector representations in a multi-dimensional space. These vector representations are used to capture the semantic or contextual meaning of the objects in a numerical format that can be easily processed by machine learning algorithms.

In some embodiments, the vector embedding functions are word embedding techniques. Word embedding techniques use natural language processing (NLP) and machine learning to represent words as dense vectors of real numbers. Word embedding techniques aim to capture the semantic and syntactic meaning of words as well as their relationships with other words in a language. Word embedding techniques include Term Frequency-Inverse Document Frequency (TF-IDF), Word2Vec, Global Vectors (GloVe), BioWordVec FastText, and Bidirectional Encoder Representation (BERT).

Each of these word embedding techniques includes salient features. The TF-IDF model is designed to give more weight to the words that are very specific to certain documents but give less weight to the words that are more general and occur across most documents. The Word2Vec model represents words in the form of dense vectors by capturing syntactic (grammar) and semantic (meaning) relationships. Given a large enough dataset, the Word2vec model provides strong estimates about a meaning of a word based on the frequency of occurrence of the word in the text. The GLOVE model is an unsupervised learning model that can be used to obtain dense word vectors like the Word2Vec model. The GLOVE model first creates a large word-context, co-occurrence matrix consisting of pairs (word, context). Each element in this matrix represents how often a word or a sequence of words occurs within the context and then applies matrix factorization to approximate this matrix. The BioWordVec fastText model is 200-dimensional word embeddings trained on PubMed and MIMIC-III data and is the extension of the original BioWordVec that provides fastText word embeddings trained using PubMed and MeSH. A subword embedding model used by the Bio WordVec fastText model better handles out of vocabulary tokens and improves the quality of the word embeddings. BERT uses encoder-only transformer architecture that learns the contextual relations between words (or subwords) in textual data and converts text into embeddings. BERT is trained on an unsupervised task of ‘Mask Language Model (MLM)’ using text corpora from BooksCorpus and English Wikipedia

In one or more embodiments, the word embedding techniques include Clinical-BERT or Self-Alignment Pretraining for Biomedical Entity Representations (SAPBERT). SAPBERT is a specialized Large Language Model (LLM) that is a pre-trained BERT model trained on Medical Entity Linking (MEL) tasks. MEL maps various entities to unified concepts in the medical knowledge graph. Word representation learning faces a significant challenge due to the existence of heterogeneous names. For example, in healthcare, terms like ‘nostril’ and ‘nare’ are used interchangeably but yield considerably different embedding representations when generated by models not specifically trained for MEL. SAPBERT works on self-alignment of biomedical entity representation such that the semantically similar entities belonging to the same concept are brought closer in the embedding space, thus forming compact clusters. SAPBERT leverages UMLS, the largest collection of biomedical concepts and synonyms and collates the synonyms from various controlled vocabularies, e.g., SNOMED CT, MeSH, Gene Ontology, RxNorm, and OMIM. SAPBERT performs better compared to other variants of BERT like Bio-BERT, Clinical-BERT with respect to the MEL challenges. The SAPBERT model can accurately capture fine-grained semantic relationships and heterogeneous naming in the biomedical domain compared to other variants of BERT. The ability of SAPBERT to handle out-of-vocabulary terms, misspelled words, and rare medical terms provides a significant advantage over other models.

In embodiments, the similarity score calculator 146 calculates a similarity between vector embeddings for entities of the model domain and vector embeddings for entities of the comparison domains. Similarity matching or similarity retrieval can be used to find items, e.g., standard codes, that are similar to a given query item, e.g., allergy free text. Similarity matching measures the similarity between a standard code and an allergy free text, based on certain features or characteristics, i.e., attributes, and then ranks the candidate standard codes by their similarity. To measure similarity, a distance measure or similarity metric is chosen. Common distance measures include Euclidean distance, cosine similarity, and Jaccard similarity. When dealing with large data sets, an index may be created to speed up the search process. An index is a data structure that organizes the data in a way that allows for efficient retrieval of similar items.

In one or more embodiments, the similarity score calculator 146 includes the Facebook AI Similarity Search (FAISS). FAISS is an open-source library developed by Facebook for efficient similarity search and clustering of high-dimensional vectors. FAISS is optimized for both CPU and GPU architectures, enabling fast and scalable similarity search operations on large datasets. FAISS supports a range of similarity metrics, including Euclidean distance, cosine similarity, inner product, and L2 distance. FAISS offers various indexing methods, including the flat index, inverted file (IVF), Hierarchical Navigable Small World (HNSW), and product quantization. Flat index uses an index built from data points without any hierarchical structure. When a search operation is performed, the distance between the query vector and all the other vectors utilized to build the index is computed and the top-n closest vectors are returned. When using IVF, a dataset is divided into clusters using a clustering algorithm (e.g., k-means). Each cluster is associated with a unique identifier. For each cluster, an inverted list is created. An inverted list is a data structure that associates a cluster identifier with the list of vectors that belong to that cluster. During indexing, each data vector is assigned to the nearest cluster centroid. This assignment is used to determine the inverted list to update with the vector's information. When performing a similarity search, the query vector is quantized to the nearest cluster centroid. FAISS then searches the inverted list associated with that cluster for potential nearest neighbors. HNSW is an algorithm for efficient similarity search in high-dimensional spaces. These indexing techniques help speed up nearest-neighbor searches in high-dimensional spaces.

In an embodiment, FAISS is combined with HNSW as the indexing approach. FAISS can be integrated with popular machine learning libraries and frameworks, such as PyTorch and TensorFlow, making it easier to incorporate similarity searches into machine learning pipelines. This may lead to significant improvements in the speed and scalability of the similarity search operations. As an open-source library, FAISS is available for developers and researchers to use, modify, and contribute to the development FAISS.

In one or more embodiments, the grouping engine 148 arranges relevant or synonymous standard codes into groups. Grouping is required to capture the synonymity of allergy concept that may be interchangeably used. The grouping engine 148 may base the groupings on TTY (for RxNorm), Category/Semantic Tag (for SNOMED CT), and/or status, e.g., active or remapped. For example, semantic clinic drugs are grouped with semantic branded drugs. Similarly generic packs are grouped with brand name packs. With respect to status, remapped codes may be grouped with active codes. Similarly, quantified codes may be grouped with active codes. The grouping logic was established through consensus among terminologists, subject matter experts, and the data science team.

In embodiments, grouping is performed using two variables with dependency on the “cardinality” variable. “S” stands for single and “M” stands for multi-cardinality. The term type of the recommended RxNorm code may be a factor in grouping of similar codes. For example,
a. IN(S) to PIN(S), BN(S), SCD(S), SBD(S), SCDC(S), SBDC(S), SNOMED-CT
b. PIN(S) to IN(S), BN(S), SCD(S), SBD(S), SCDC(S), SBDC(S), SNOMED-CT
c. MIN (M) to BN (M), SCD (M), SBD (M), SCDC (M), SBDC (M), SNOMED-CT
d. BN(S) to IN(S), PIN(S), SCD(S), SBD(S), SCDC(S), SBDC(S), SNOMED-CT
e. BN (M) to MIN (M), SCD (M), SBD (SM), SCDC (M), SBDC (M), SNOMED-CT
f. SNOMED codes that do not fall in a-e groups have their own groups with single codes.

The status, i.e., remapped, quantified and active, may also be a factor in grouping of similar codes. For example, when the recommended text is “tramadol” having an RxNorm code 10689 with term type as IN and cardinality as “S”, the grouping includes:
PIN:—82110 (S)|tramadol hydrochloride;
BN: 1148479 (S)|ConZip, 220606 (S)|Ultram, 2395809 (S)|Qdolo;
SCDC: 836484 (S)|tramadol hydrochloride 1 MG/ML, 2395807 (S)|traMADol hydrochloride 5 MG/ML, 2670389 (S)|tramadol hydrochloride 25 MG etc. (Total 13 SCDCs related)
SBDC: 2395810 (S)|traMADol hydrochloride 5 MG/ML [Qdolo], 835604 (S)|traMADol hydrochloride 50 MG [Ultram] etc. (Total 7 SBDCs related)
SCD: 849561 (S)|12 HR tramadol hydrochloride 150 MG Extended Release Oral Tablet, 1148478 (S)|24 HR traMADol hydrochloride 100 MG Extended Release Oral Capsule etc. (Total 21 SCD related)
SBD: 1148482 (S)|ConZip 100 MG 24 HR Extended Release Oral Capsule, 835605 (S)|Ultram 50 MG Oral Tablet etc. (Total 21 SBD related)
SNOMED-CT: 108507005|Product containing tramadol (medicinal product), 386858008|Tramadol (substance).
The resulting final grouping for “tramadol” is [′10689′, ‘82110’, ‘1148479’, ‘220606’, ‘2395809’, ‘836484’, ‘2395807’, ‘2670389’, ‘2395810’, ‘835604’, ‘849561’, ‘1148478’, ‘1148482’, ‘835605’, ‘108507005’, ‘386858008’, <and other codes that are not shown here as the list is long>].

When the recommended text is part of a SNOMED CT code, then all the other synonymous SNOMED-CT codes and relevant RxNorm Codes based on any of the above logic (TTYs and SNOMED CT) are grouped together. For example, when the recommended text is “Taurolidine” having a SNOMED-CT code 281000220103, the group includes SNOMED CT code 1287715004|Product containing taurolidine (medicinal product)′; and IN: 2670453 (S)|taurolidine. The final grouping for “Taurolidine” is [′281000220103′, ‘1287715004’, ‘2670453’].

In one or more embodiments, the selection engine 150 presents candidate standard codes for mapping to target allergy free text to the user interface 106 based on the similarity values 128 provided by the similarity score calculator 146. The selection engine 150 may present an “N” number of candidate standard codes for mapping to a target allergy free text. The “N” number of candidate standard codes may be ranked by the similarity values between the vector embeddings of the candidate standard codes and the vector embedding of the allergy free text. Alternatively, the selection engine 150 presents every standard codes having a similarity measure above a threshold.

In one or more embodiments, the deduplication engine 152 is a software component or system designed to identify and remove duplicate allergy event entries from a patient's allergy list or medication history. Duplicate allergy event entries can occur due to various factors, such as discrepancies in data entry.

The deduplication engine 152 analyzes the allergy list to identify duplicate entries based on predefined criteria or rules. Common criteria for duplicate detection include allergen name and allergy type. The deduplication engine 152 compares allergy entries against each other to identify potential duplicates or near-duplicates.

In one or more embodiments, the deduplication engine 152 employs matching algorithms and similarity analysis techniques to compare allergy entries and determine their similarity or equivalence. Once duplicate allergy entries are identified, the engine applies deduplication strategies to resolve duplicates and consolidate redundant information. Deduplication strategies may involve merging duplicate entries, updating existing entries with the most current information, or flagging duplicates for manual review by healthcare providers.

In embodiments, the deduplication engine 152 prioritizes duplicate allergies entries based on severity, clinical relevance, or potential patient harm. The deduplication engine 152 provides decision support tools and recommendations to assist healthcare providers in resolving duplicates and making informed decisions about allergy management. This may include alerts, notifications, or suggestions for medication reconciliation and rationalization. The deduplication engine 152 integrates with clinical systems, medication management tools, and EHR systems to facilitate seamless deduplication workflows. The deduplication engine 152 may present deduplicated allergy lists to healthcare providers within their existing workflows, allowing users to review, validate, and finalize allergy reconciliations efficiently.

In one or more embodiments, user interface 106 refers to hardware and/or software configured to facilitate communications between a user and the synchronization engine 104. User interface 106 renders user interface elements and receives input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of user interface 106 are specified in different languages. The behavior of user interface elements is specified in a dynamic programming language, such as JavaScript. The content of user interface elements is specified in a markup language, such as hypertext markup language (HTML) or XML User Interface Language (XUL). The layout of user interface elements is specified in a style sheet language, such as Cascading Style Sheets (CSS). Alternatively, user interface 106 is specified in one or more other languages, such as Java, C, or C++.

In one or more embodiments, external sources 108 for patient records include health information exchange (HIE) 154 and healthcare databases 156.

In one or more embodiments, HIE 154 refers to the secure electronic sharing of health-related information between healthcare organizations, healthcare professionals, patients, and other authorized parties. HIE 154 enables the seamless exchange of patient health information, including medical records, test results, medication histories, treatment plans, and other clinical data, across different healthcare settings and systems. The primary goal of HIE 154 is to improve care coordination, enhance patient outcomes, and optimize healthcare delivery by ensuring that relevant and timely information is accessible to healthcare providers at the point of care. HIE systems adhere to interoperability standards and specifications that define how health information is structured, transmitted, and accessed. Common standards include HL7 (Health Level Seven), FHIR (Fast Healthcare Interoperability Resources), CCD (Continuity of Care Document), and DICOM (Digital Imaging and Communications in Medicine). HIE networks consist of participating healthcare organizations, providers, laboratories, pharmacies, payers, and other entities that contribute and exchange health information within the network. Participants may include hospitals, clinics, physician practices, long-term care facilities, public health agencies, and insurers.

In one or more embodiments, HIE systems employ secure data exchange infrastructure, such as health information exchange platforms, networks, and interfaces, to facilitate the transmission of health information between disparate systems and organizations. This infrastructure may include electronic interfaces, secure messaging protocols, health information service providers (HISPs), and data repositories. HIE systems incorporate mechanisms for obtaining patient consent and authorization for the sharing of their health information. Patients have the right to control who can access their health information and can specify their preferences regarding data sharing and disclosure. HIE initiatives implement robust data governance and security measures to protect the privacy, confidentiality, and integrity of patient health information. This includes encryption, access controls, authentication mechanisms, audit trails, and compliance with regulatory requirements such as HIPAA (Health Insurance Portability and Accountability Act). HIE systems support query-based exchange, allowing authorized healthcare providers to access patient health information from other participating organizations as needed. Providers can query the HIE network to retrieve relevant clinical data, such as medical history, allergies, medications, and lab results, to support clinical decision-making and care coordination. HIE systems facilitate directed exchange of health information between specific providers or organizations involved in the care of a shared patient. This enables targeted sharing of information, such as referrals, care transitions, and care coordination activities, while maintaining patient privacy and confidentiality.

In one or more embodiments, healthcare databases 156 refer to repositories of electronic health information that store, organize, and manage a wide range of healthcare-related data. These databases play a crucial role in various healthcare functions, including patient care, clinical research, population health management, and healthcare administration.

In one or more embodiments, the healthcare databases 156 include EHR databases, clinical data warehouses, and research databases. EHR databases store comprehensive electronic health records for individual patients, including demographic information, medical history, medications, allergies, lab results, imaging studies, clinical notes, and treatment plans. EHRs support clinical documentation, care coordination, and patient management across different healthcare settings. Clinical data warehouses aggregate and integrate data from multiple sources, such as EHRs, laboratory systems, radiology systems, pharmacy systems, and billing systems, into a centralized repository. These databases enable healthcare organizations to perform analytics, reporting, and research on large datasets to support clinical decision-making, quality improvement, and population health management. Research databases store clinical research data, including patient enrollment information, study protocols, clinical trial data, and research findings. These databases support clinical research initiatives, observational studies, comparative effectiveness research, and post-market surveillance of medical products.

In one or more embodiments, the healthcare databases 156 include registries, administrative databases, and pharmacy databases. Registries are specialized databases that collect and maintain data on specific populations, diseases, conditions, or procedures. Examples include cancer registries, diabetes registries, transplant registries, and immunization registries. Registries facilitate disease surveillance, epidemiological research, and outcomes monitoring to improve healthcare delivery and public health. Administrative databases contain administrative and billing data generated by healthcare organizations, insurers, and government agencies. These databases capture information such as patient demographics, insurance coverage, healthcare services rendered, reimbursement claims, and healthcare costs. Administrative databases support healthcare financing, reimbursement, policy analysis, and resource allocation. Pharmacy databases store data related to medication dispensing, prescription orders, medication administration, and medication usage patterns. These databases provide insights into medication adherence, prescribing trends, drug utilization, and medication safety to support medication management and quality improvement initiatives.

### 3. Presenting Recommendations of Candidate Standard Codes for Mapping to Unmapped Allergy Codes to Facilitate Allergy Event Deduplication

FIGS. 2A-2C illustrate an example set of operations for recommending standard codes for mapping to unmapped allergy codes to facilitate allergy event deduplication in accordance with one or more embodiments. One or more operations illustrated in FIGS. 2A-2C may be modified, rearranged, or omitted all together. Accordingly, the particular sequence of operations illustrated in FIGS. 2A-2C should not be construed as limiting the scope of one or more embodiments.

One or more embodiments access patient allergy data from healthcare databases for the patient, the patient allergy data including allergy data associated with patient allergy events (Operation 202). When the system detects a trigger for synchronizing a patient allergy record, e.g., scheduling of an appointment or admittance to a hospital, the system queries the relevant healthcare databases to retrieve the patient's allergy data. Retrieval of patient allergy data may involve querying electronic health record (EHR) systems, pharmacy databases, medication management systems, or other clinical databases that store allergy-related information. The queried data is extracted from the healthcare databases, focusing specifically on allergy-related information. Allergy-related information includes details such as allergen names and allergen types.

One or more embodiments crossmap the codes associated with the allergy events with standard codes (Operation 204). Cross-mapping allergy codes associated with allergy events to standard codes involves translating or converting the allergy codes used in a specific system or database to standardized codes recognized across healthcare settings. The system applies the mapping algorithm or rules to cross-map the allergy codes associated with allergy events to standard codes. Mapping involves matching each source code to the most appropriate standard code or codes based on the defined mappings and criteria.

One or more embodiments determine if any of the unmapped allergy events includes allergy free text (Operation 206). To determine if any of the unmapped allergy events include allergy free text, the system inspects the allergy event data and identifies instances where the allergy information is not mapped to standard codes and instead appears as structured or unstructured free-text data.

One or more embodiments remove unmapped allergy events not associated with allergy free text from patient allergy data (Operation 208). When an unmapped allergy event is not associated with a standard code and does not include allergy free text, the unmapped allergy event may be removed from the patient allergy data. Alternatively, the unmapped allergy events may be annotated and presented to a healthcare provider as unmapped or unknown allergy events.

One or more embodiments generate a plurality of vectors embeddings for the unmapped allergy codes using the allergy free text associated with the corresponding unmapped allergy codes (Operation 210). The system uses vector embedding techniques, e.g., natural language processing (NLP), to represent the text data as numerical vectors in a high-dimensional space. The allergy free text may be preprocessed to clean and standardize the text data. Preprocessing may involve steps such as tokenization, lowercasing, removing punctuation, stop word removal. Pre-trained word embeddings models, e.g. SABERT, are used to represent words in the text data as dense numerical vectors. These word embeddings capture semantic relationships between words based on their context and distributional patterns in large text corpora. Individual word embeddings may be combined to generate sentence-level embeddings for the allergy free text associated with each unmapped allergy code. Various techniques can be used for this purpose, including averaging word embeddings, weighted averaging based on term frequency-inverse document frequency (TF-IDF). Each unmapped allergy code is represented as a numerical vector or vector embedding in the high-dimensional space, capturing the semantic meaning of the corresponding allergy free text. These vector embeddings will be used for various downstream tasks such as similarity comparison, clustering, classification, or information retrieval.

One or more embodiments access a plurality of standard codes, each standard code corresponding to a set of attributes (Operation 212). The standard codes may be accessed through online resources, licensing the data from a vendor or organization, or utilizing open-access datasets provided by standardization bodies or government agencies. Using tools, APIs, or databases provided by the standardized coding system to query or browse the available allergy codes. For each standard event code, the system retrieves the corresponding attributes associated with the code. The structured data may include information such as drug names, allergen names, allergen types, and other relevant clinical attributes.

In embodiments, the plurality of standard codes are from a first set of standard codes and a second set of standard codes. The first set of standard codes may better describe medications to which the patient may be allergic. The second set of standard codes may better describe non-medications or substances, e.g., food, to which the patient may be allergic. For example, the first set of standard codes may be RxNorm while the second set of standard codes may be SNOMED CT.

One or more embodiments generate a plurality of vector embeddings for the plurality of standard codes using text of the attributes corresponding to each standard event code (Operation 214). Generating the plurality of vector embeddings for the plurality of standard codes includes applying the same vector embedding technique as applied to the allergy free text to the text of the attributes associated with the respective standard codes. The text may be preprocessed in the same manner as the text of the attributes associated with the respective standard codes. Individual word embeddings may be combined to generate sentence-level embeddings for the text of the attributes associated with each standard code.

One or more embodiments compute a similarity metric for a first target vector embedding and a first vector embedding of a first standard code of the plurality of standard codes (Operation 216). To compute the similarity metric between the first target vector embedding and the vector embedding of a standard code the system uses a similarity measure, e.g., cosine similarity or Euclidean distance.

One or more embodiments determine if the similarity metric for the first target vector embedding and the first vector embedding meet a threshold (Operation 218). The system determines if the similarity metric for the first target vector embedding and the first vector embedding meet the threshold by comparing the similarity metric to a threshold. The threshold depends on the similarity measure used and the level of similarity consider significant. For cosine similarity, the value ranges from −1 to 1, where a value closer to 1 indicates higher similarity. In embodiments, a similarity metric exceeding 0.9 meets the threshold.

One or more embodiments present the first standard code as a candidate standard code for mapping to the allergy free text (Operation 220). When the similarity metric of the first target vector embedding and the first vector embedding meet or exceed the threshold, the system presents the first standard code for mapping to the first unmapped allergy code.

One or more embodiments refrain from presenting the first standard code as a candidate for mapping to the first unmapped allergy code (Operation 222). When the similarity metric of the first target vector embedding and the first vector embedding fail to meet the threshold, the system refrains from presenting the first standard code for mapping to the first unmapped allergy code.

One or more embodiments create a first grouping comprising the first standard code with one or more similar standard codes (Operation 224). The system identifies one or more standard codes that are relevant or synonymous to the first standard code. When RxNorm codes are used as the standard codes, the grouping may be according to TTY or status. For example, IN(S) may be grouped with PIN(S), BN(S), SCD(S), SBD(S), SCDC(S), SBDC(S), SNOMED-CT. Similarly, remapped codes may be grouped with active codes, and quantified codes may be mapped with active codes. When SNOMED CT codes are used as the standard codes, the groupings may be according to semantic tags or status. When both RxNorm codes and SNOMED CT codes are used, SNOMED CT codes may be preferred over RxNorm codes when the allergy data is associated with food and/or substances and RxNorm codes may be preferred over SNOMED CT codes when the allergy data is associated with medications.

One or more embodiments present the first grouping to a user as a candidate grouping for mapping to the first unmapped allergy code (Operation 226). The groupings are then presented to the user as candidates for the first unmapped allergy code. The groupings may be presented in a user interface that includes the allergy free text associated with the first unmapped allergy code, the candidate standard code, the name associated with the candidate standard code, and the similarity metric or score. The grouping presented in the user interface may also include one or more standard codes similar to the candidate standard code, and the names associated with the one or more standard codes similar to the candidate standard code. Each grouping may be provided with a reference number or code for future use.

One or more embodiments receive user input confirming the standard code associated with the first grouping for mapping to the first unmapped allergy code (Operation 228). The user may select the standard code from the user interface for mapping to the first unmapped allergy code by selecting a standard code from the list, entering the standard code manually, or providing any additional comments or feedback. Alternatively, the system may automatically confirm a mapping of a standard code with an allergy free text when the similarity metric is above a threshold.

In embodiments, when the candidate standard codes included in a grouping are both RxNorm codes and SNOMED CT, the grouping is represented by TTY and status in the following order: active-IN, active-MIN, active-PIN, active-BN, active-SNOMED, remapped-IN, remapped-MIN, remapped-PIN, and remapped-BN. In this manner, active-IN receives the highest priority and active-SNOMED and remapped-IN receive the lowest priority.

In one or more embodiments, the user input confirming selection of a standard code for mapping to the allergy free text is saved as a potential mapping for review by a terminologist. After the mapping is confirmed by the terminologist, the mapping may be added to the database of allergy free text mapped to standard codes. In this manner, the previously unmapped allergy code associated with allergy free text becomes a mapped allergy code as the allergy free text is mapped to a standard code.

One or more embodiments deduplicate patient allergy events associated with the mapped codes (Operation 230). The system uses the selected standard code to arrange similar or like patient allergy events into buckets. Duplicate patient allergy events, e.g., patient allergy events having the same standard code or a standard code from the same group as other patient allergy events, may be removed. Alternatively, the duplicate patient allergy events may be highlighted or otherwise annotated to indicate a potential that the patient allergy event is a duplicate.

One or more embodiments present deduplicated patient allergy events to a healthcare provider (Operation 232). The patient allergy events may be presented to the healthcare provider in a longitudinal record or other visual form. The longitudinal record may include a first column of verified patient allergy events and a second column of potential duplicates. The healthcare provider may provide indication confirming the mapping of standard codes to allergy free text. The healthcare provider may also have the ability to provide feedback as to why confirmation was not provided for the mapping.

### 4. Example Synchronization Operation

A detailed example is described below for purposes of clarity. Components and/or operations described below should be understood as one specific example which may not be applicable to certain embodiments. Accordingly, components and/or operations described below should not be construed as limiting the scope of any of the claims.

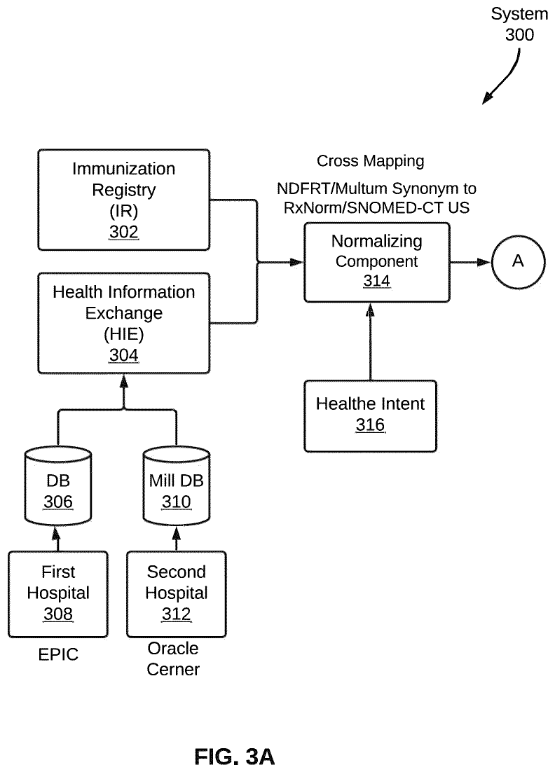

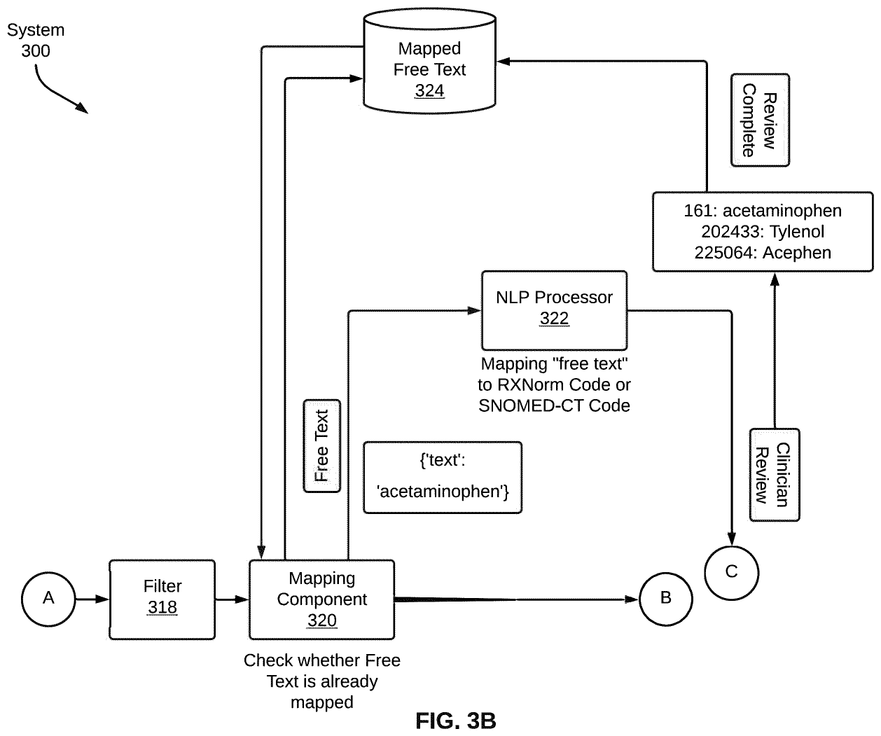

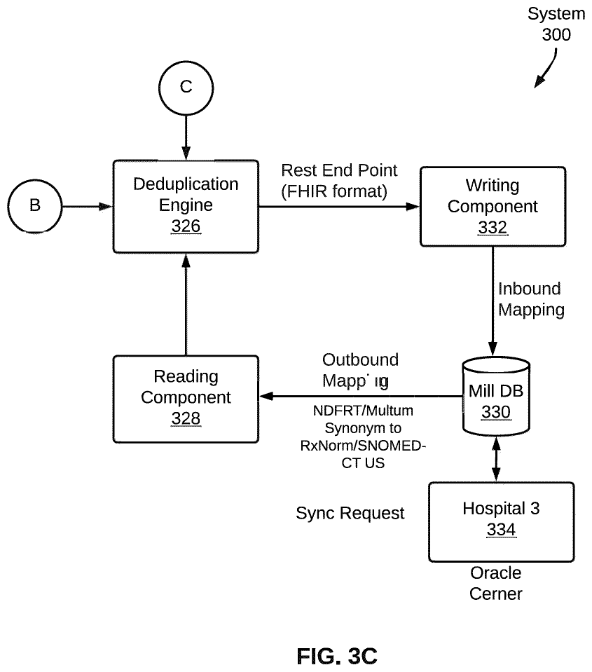

FIG. 3A illustrates a record retrieval portion of a system 300 for synchronizing a patient allergy record. The record retrieval portion of system 300 retrieves patient allergy information from patient records stored in the immunization registry 302 and/or from an HIE 304. As shown, the HIE 304 receives patient allergy information from a database 306 associated with a first hospital 308. The first hospital 308 uses a first EHR system, e.g., EPIC. The HIE 304 may also receive patient allergy information from a Millennium Database 310 associated with a second hospital 312. The second hospital 312 uses a second EHR system, e.g., Oracle Cerner.

Patient information is provided to a normalizing component 314. The normalizing component 314 may also receive patient data from Healthe Intent 316. Healthe Intent 316 is a population health management platform developed by Cerner to assist healthcare organizations manage the health of a large group of people, like a community or an entire patient base. The normalizing component 314 identifies patient allergy events, crossmaps patient allergy events provided as proprietary codes to standard codes.

FIG. 3B illustrates a mapping portion of the system 300. The mapping portion of the system 300 includes a filter 318 for filtering the patient allergy data received from the normalizing component 314. Alternatively, filter 318 is part of normalizing component 314. The filter 318 identifies allergy codes including allergy free text. A mapping component 320 determines if the allergy free text has already been mapped to a standard code.

When the allergy free text has not been previously mapped, the allergy free text is provided to an NLP processor 322. The NLP processor 322 includes various components for preprocessing the text and generating one or more vector embeddings for the allergy free text. The NLP processor 322 may also generate vector embeddings for standard codes and determine similarity metrics for the vector embeddings for the allergy free text and the vector embeddings for the standard codes. The NLP processor 322 may also include a grouping component for grouping similar standard codes with the recommended standard codes. A selection component of the NLP processor 322 allows for selection of a recommended standard code by a healthcare provider.

A terminologist reviews the selection of the standard code for mapping to the allergy free text. Upon determination by the terminologist that the standard code selected for mapping to the allergy free text is accurate, the mapping of the standard code to the allergy free text is stored in a database of mapped allergy free text 324. The mapped allergy free text 324 may be used by the mapping component 320 during future mapping operations of allergy free text.

FIG. 3C illustrates a deduplication portion of system 300 for synchronizing patient allergy data. The mapped patient allergy events that were originally associated with a standard code or that were mapped to a standard code are received by a deduplication engine 326, as indicated at “B”. The deduplication engine 326 also receives the standard codes selected for association with the allergy codes that included allergy free text, as indicated at “C”. The deduplication engine 326 may also receive standard codes from a reading component 328. The reading component 328 receives patient allergy data from a Millenium DB 330 associated with a third hospital 334 that initiated the synchronization request. The third hospital 334 uses an Oracle Cerner EHR system.

The deduplication engine 326 identifies and removes duplicate patient allergy events. The deduplicated patient allergy events are passed to a writing component 332 that writes the patient allergy events to the Mill DB 330. A healthcare provider at the third hospital 334 access the deduplicated patient allergy data from the Mill DB 330.

### 5. Example Synchronization System

A detailed example is described below for purposes of clarity. Components and/or operations described below should be understood as one specific example which may not be applicable to certain embodiments. Accordingly, components and/or operations described below should not be construed as limiting the scope of any of the claims.

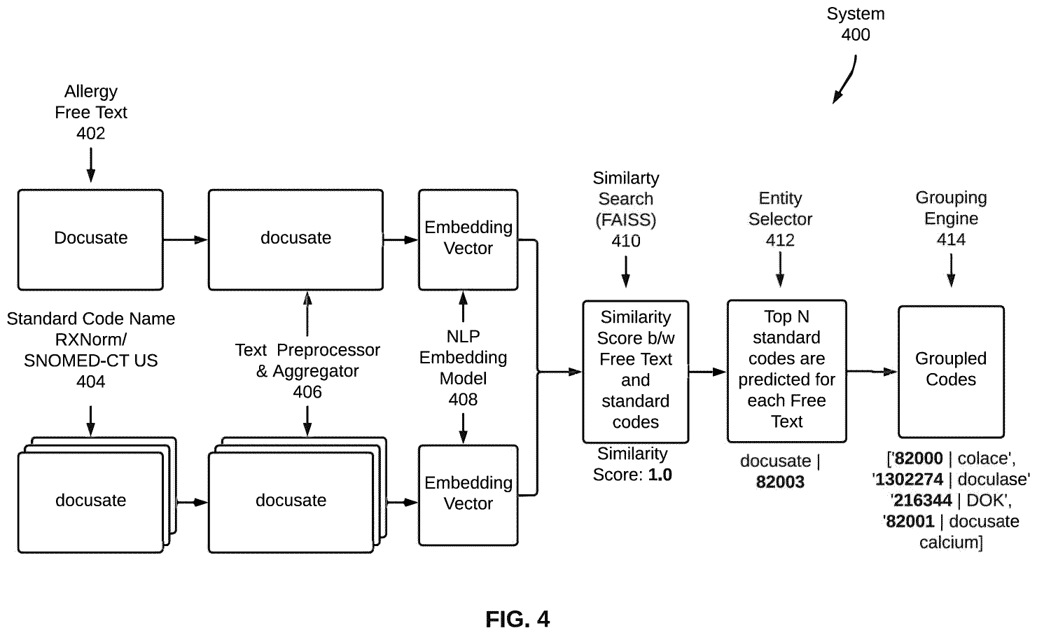

FIG. 4 illustrates operations for providing recommendations for mapping of standard codes to allergy free text. Initially, a patient allergy event associated with allergy free text 402 is identified. The allergy free text 402 includes “docusate”. The text for one or more attribute fields of standard codes, e.g., RxNorm and SNOMED, are identified. As shown, the attribute field for the standard code name 404 are RXNorm/SNOMED CT code names. The standard code names 404 includes “docusate”.

A text aggregator and preprocessor 406 is applied to the allergy free text 402. Similarly, the text aggregator and preprocessor 406 is applied to the text of the standard code names 404. As shown, the text preprocessing includes lower casing the text.

An NLP embedding model 408 is applied to the preprocessed and aggregated text of the allergy free text 402. The NLP embedding model 408 generates an embedding vector for the allergy free text. Similarly, the NLP embedding model 408 is applied to the text of the standard code names 404 to generate an embedding vector for the standard codes associated with the standard codes names 404.

A similarity search 410 calculates a similarity score between the embedding vector of the allergy free text and the embedding vector of the standard codes associated with the standard code names 404.

An entity selector 412 presents the top “N” standard codes for mapping to the allergy free text. The top “N” Standard codes are ranked based on the similarity scores computed by the similarity search 410, the term type of the standard codes, the source of the standard codes, and the status of the standard codes.

A grouping engine 414 provides one or more additional standard codes for grouping with the recommended standard codes. The additional standard codes are synonymous or of relevant scope to the recommend standard code.

A user interface 416 displays the final recommendations of standard codes for mapping to allergy free text. The final recommendations of standard codes may include a table listing the standard code name, the similarity score, and the one or more additional standard codes.

### 6. Recommendation Interface

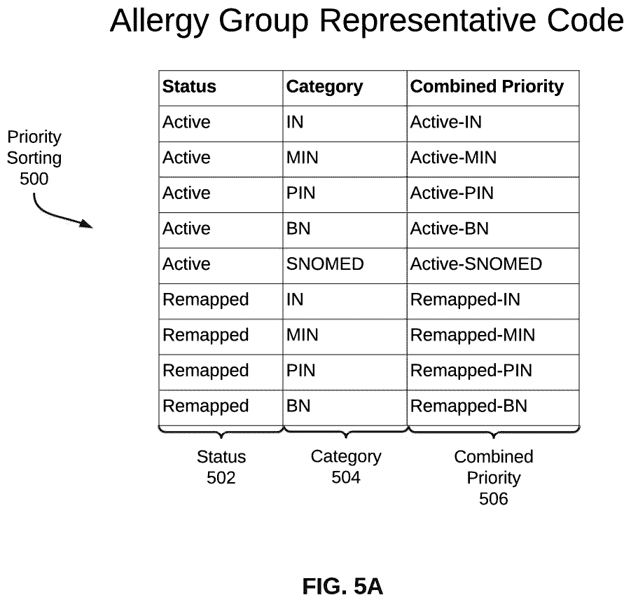

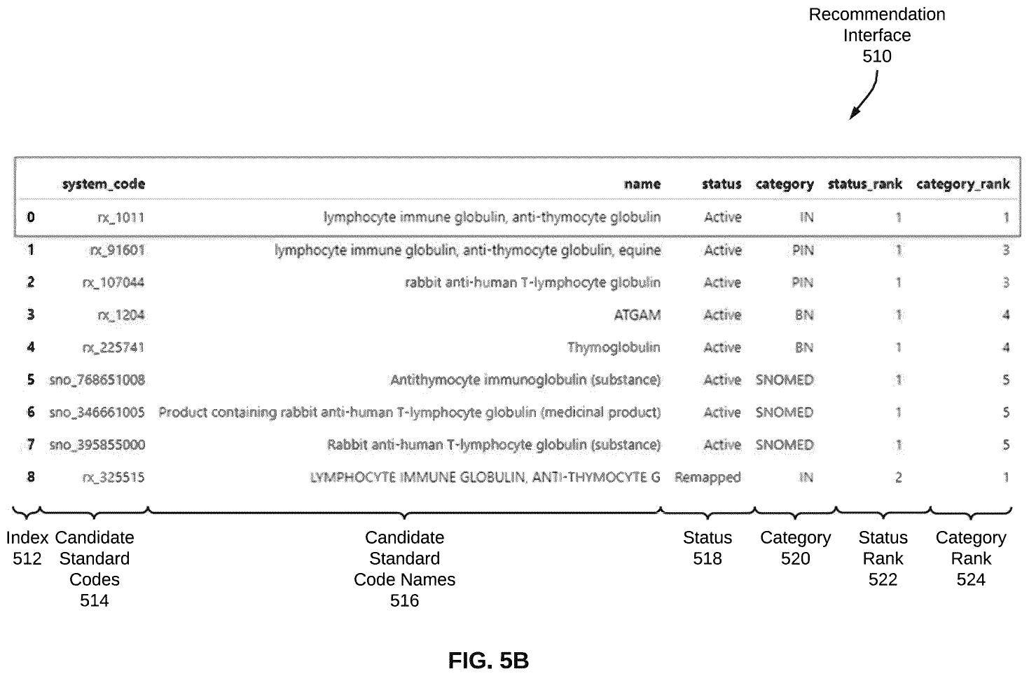

FIG. 5A illustrates an example of a priority sorting 500 for standard codes associated with allergy events in accordance with one or more embodiments. The priority sorting 500 may display information in a table format for easy viewing. The priority sorting 500 includes status 502 of the standard codes, category 504 of the standard codes, and combined priority 506 of the standard codes. As shown, active standard codes are prioritized ahead of remapped standard codes and active RxNorm codes are prioritized ahead of active SNOMED CT codes.

FIG. 5B illustrates an example recommendation interface 510 in accordance with one or more embodiments. The recommendation interface 510 may display information in a table format for easy viewing. The recommendation interface 510 provides recommendations of standard codes, i.e., RxNorm and SNOMED CT, for allergy free text.

The recommendation interface 510 provides a table including an index 512, candidate standard code 514, candidate standard code name 516, status 518 of the candidate standard codes, category 520 of the candidate codes, status rank 522 of the candidate standard codes, and category rank 524 of the standard codes. The index 512 provides a ranking of the candidate standard codes 514. The rankings may be according to similarity scores. The candidate standard codes 514 are the RxNorm code or SNOMED CT code. The candidate standard code names 516 are the RxNorm code name or SNOMED CT code name for the respective candidate standard codes 514. Status 518 indicates whether a candidate standard code 514 is active or remapped. Category 520 identifies a category for RxNorm codes. Status rank 522 for all active codes is “1” and for remapped codes is “2”. Category rank 524 is determined by the priority sorting 500 shown in FIG. 5B.

### 7. Practical Application; Improvements & Advantages

Deduplication in healthcare data serves several important purposes, contributing to the overall quality, integrity, and efficiency of healthcare information systems. Deduplication ensures that each patient has a unique and accurate record, reducing the risk of administering treatments or medications that may illicit an allergic reaction in the patient. Deduplication processes help maintain the accuracy and integrity of patient information, supporting better decision-making by healthcare providers. Healthcare providers are able to access a complete and consolidated view of a patient's medical history, allergies, and treatments without the interference of redundant or conflicting information. By implementing deduplication processes, healthcare organizations can provide physicians with a more reliable and consistent patient history. This, in turn, may reduce the burden on healthcare providers related to correcting and reconciling conflicting information, allowing the healthcare providers to focus more on patient care.

Deduplication contributes to the reliability of healthcare analytics, enabling healthcare organizations to derive insights from clean and consolidated data. Deduplicating healthcare data helps optimize resource utilization, reducing storage requirements and improving the overall performance of information systems. Deduplication supports interoperability between different healthcare systems. When patient records are consistently unique and accurate, exchanging information seamlessly between various healthcare providers, systems, and organizations becomes easier. Improved interoperability can help reduce the frustration that physicians may experience when dealing with disparate and incompatible data sources. Having a more integrated and seamless exchange of information can contribute to a smoother and less stressful workflow. Patients benefit from deduplication as it ensures that their medical information is accurate and up-to-date. This, in turn, contributes to a better patient experience, as healthcare providers can offer more personalized and effective care.

### 8. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

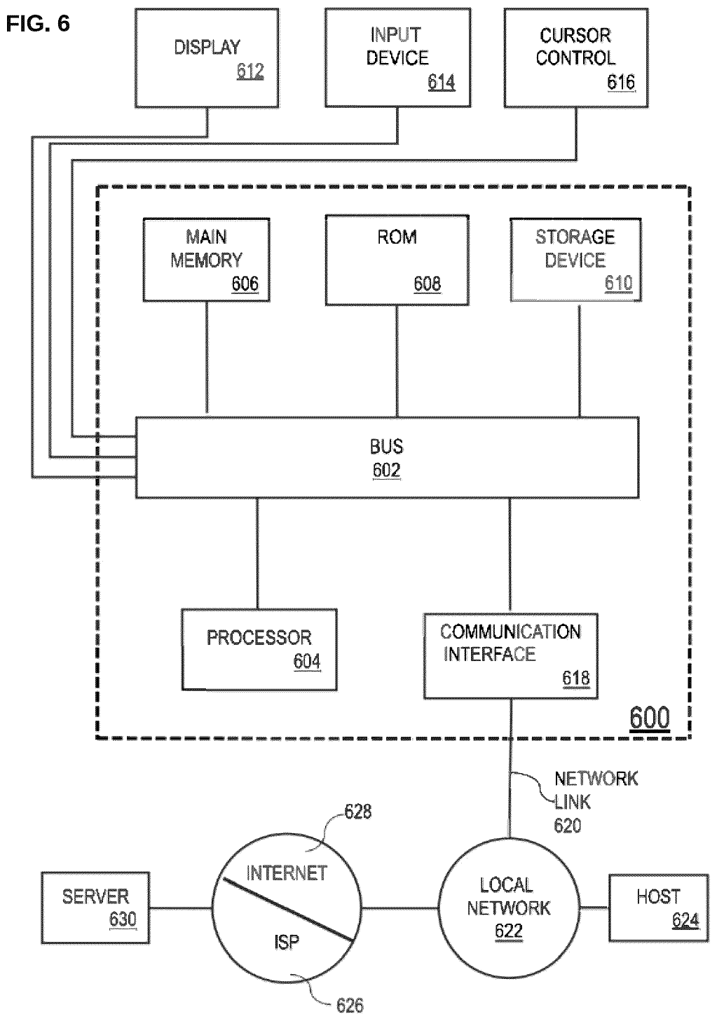

For example, FIG. 6 is a block diagram that illustrates a computer system 600 upon which an embodiment of the disclosure may be implemented. Computer system 600 includes a bus 602 or other communication mechanism for communicating information, and a hardware processor 604 coupled with bus 602 for processing information. Hardware processor 604 may be, for example, a general purpose microprocessor.

Computer system 600 also includes a main memory 606, such as a random access memory (RAM) or other dynamic storage device, coupled to bus 602 for storing information and instructions to be executed by processor 604. Main memory 606 also may be used for storing temporary variables or other intermediate information during execution of instructions to be executed by processor 604. Such instructions, when stored in non-transitory storage media accessible to processor 604, render computer system 600 into a special-purpose machine that is customized to perform the operations specified in the instructions.

Computer system 600 further includes a read only memory (ROM) 608 or other static storage device coupled to bus 602 for storing static information and instructions for processor 604. A storage device 610, such as a magnetic disk, optical disk, or a Solid State Drive (SSD) is provided and coupled to bus 602 for storing information and instructions.

Computer system 600 may be coupled via bus 602 to a display 612, such as a cathode ray tube (CRT), for displaying information to a computer user. An input device 614, including alphanumeric and other keys, is coupled to bus 602 for communicating information and command selections to processor 604. Another type of user input device is cursor control 616, such as a mouse, a trackball, or cursor direction keys for communicating direction information and command selections to processor 604 and for controlling cursor movement on display 612. This input device typically has two degrees of freedom in two axes, a first axis (e.g., x) and a second axis (e.g., y), that allows the device to specify positions in a plane.

Computer system 600 may implement the techniques described herein using customized hard-wired logic, one or more ASICs or FPGAs, firmware and/or program logic which in combination with the computer system causes or programs computer system 600 to be a special-purpose machine. According to one embodiment, the techniques herein are performed by computer system 600 in response to processor 604 executing one or more sequences of one or more instructions contained in main memory 606. Such instructions may be read into main memory 606 from another storage medium, such as storage device 610. Execution of the sequences of instructions contained in main memory 606 causes processor 604 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 610. Volatile media includes dynamic memory, such as main memory 606. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 602. Transmission media can also take the form of acoustic or light waves, such as those generated during radio-wave and infra-red data communications.

Various forms of media may be involved in carrying one or more sequences of one or more instructions to processor 604 for execution. For example, the instructions may initially be carried on a magnetic disk or solid state drive of a remote computer. The remote computer can load the instructions into its dynamic memory and send the instructions over a telephone line using a modem. A modem local to computer system 600 can receive the data on the telephone line and use an infra-red transmitter to convert the data to an infra-red signal. An infra-red detector can receive the data carried in the infra-red signal and appropriate circuitry can place the data on bus 602. Bus 602 carries the data to main memory 606, from which processor 604 retrieves and executes the instructions. The instructions received by main memory 606 may optionally be stored on storage device 610 either before or after execution by processor 604.

Computer system 600 also includes a communication interface 618 coupled to bus 602. Communication interface 618 provides a two-way data communication coupling to a network link 620 that is connected to a local network 622. For example, communication interface 618 may be an integrated services digital network (ISDN) card, cable modem, satellite modem, or a modem to provide a data communication connection to a corresponding type of telephone line. As another example, communication interface 618 may be a local area network (LAN) card to provide a data communication connection to a compatible LAN. Wireless links may also be implemented. In any such implementation, communication interface 618 sends and receives electrical, electromagnetic or optical signals that carry digital data streams representing various types of information.

Network link 620 typically provides data communication through one or more networks to other data devices. For example, network link 620 may provide a connection through local network 622 to a host computer 624 or to data equipment operated by an Internet Service Provider (ISP) 626. ISP 626 in turn provides data communication services through the world wide packet data communication network now commonly referred to as the “Internet” 628. Local network 622 and Internet 628 both use electrical, electromagnetic or optical signals that carry digital data streams. The signals through the various networks and the signals on network link 620 and through communication interface 618, which carry the digital data to and from computer system 600, are example forms of transmission media.

Computer system 600 can send messages and receive data, including program code, through the network(s), network link 620 and communication interface 618. In the Internet example, a server 630 might transmit a requested code for an application program through Internet 628, ISP 626, local network 622 and communication interface 618.

The received code may be executed by processor 604 as it is received, and/or stored in storage device 610, or other non-volatile storage for later execution.

### 9. Miscellaneous; Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. One or more non-transitory computer readable media comprising instructions which, when executed by one or more hardware processors, cause performance of operations comprising:
accessing a plurality of standard codes, each standard code being mapped to a corresponding set of attributes;
generating a plurality of vector embeddings corresponding respectively to the plurality of standard codes, wherein generating the plurality of vector embeddings comprises:
applying a machine learning model to text of a first set of attributes associated with a first standard code of the plurality of standard codes for a first patient allergy event, to generate a first vector embedding;
accessing patient allergy data of a patient from one or more sources, wherein the patient allergy data comprises a target unmapped allergy code corresponding to a target allergy event, the target unmapped allergy code comprises allergy free text;
applying a machine learning model to the allergy free text of the target unmapped allergy code to generate a target vector embedding for the target unmapped allergy code;
computing a similarity measure for the target vector embedding and each of the plurality of vector embeddings to generate a plurality of similarity measures, the plurality of similarity measures comprise:
a first similarity measure for the target vector embedding and the first vector embedding; and
based at least on the first similarity measure, presenting the first standard code as a candidate standard code for mapping to the target unmapped allergy code.

2. The one or more non-transitory computer readable media of claim 1, wherein the plurality of standard codes comprise:
a. a first set of standard codes provided by a first entity, and
b. a second set of standard codes provided by a second entity, the first set of standard codes being different from the second set of standard codes, and the first entity being different from the second entity.

3. The one or more non-transitory computer readable media of claim 2, wherein the first set of standard codes comprises RxNorm and set of standard codes comprises SNOMED CT.

4. The one or more non-transitory computer readable media of claim 2, wherein a first candidate standard code from the first set of standard codes is presented above a candidate standard code from the second set of standard codes.

5. The one or more non-transitory computer readable media of claim 1, wherein generating the plurality of vector embeddings further comprises:
applying the machine learning model to text of a second set of attributes mapped to a second standard code, of the plurality of standard codes, for a second allergy event, to generate a second vector embedding;
wherein the plurality of similarity measures further comprise:
a second similarity measure for the target vector embedding and the second vector embedding;
wherein the operations further comprise:
based at least on the second similarity measure, refraining from presenting the second standard code as a candidate standard code for mapping to the target unmapped allergy code.

6. The one or more non-transitory computer readable media of claim 1, wherein the patient allergy data further comprises a second allergy event, wherein a second standard code corresponds to the second allergy event, wherein the operations further comprise:
identifying that the second standard code associated with the second allergy event is the same as first standard code associated with the target allergy event; and
removing one of the target or second allergy event from the patient allergy data as duplicative of the other of the target or second allergy event.

7. The one or more non-transitory computer readable media of claim 1, the operations further comprising,
identifying one or more standard codes that are similar to the first standard code;
generating a first grouping comprising the first standard code and the one or more similar standard codes,
wherein presenting the first standard code further comprises presenting the first grouping.

8. The one or more non-transitory computer readable media of claim 7, wherein the patient allergy data further comprises a second allergy event, wherein a second standard code corresponds to the second allergy event, wherein the operations further comprise:
identifying that the second standard code associated with the second allergy event corresponds to one of the one or more similar standard codes in the first grouping; and
removing one of the target or second allergy event from the patient allergy data as duplicative of the other of the target or second allergy event.

9. The one or more non-transitory computer readable media of claim 1, wherein the first similarity measure comprises a weighted cosine similarity measure for the target vector embedding and the first vector embedding.

10. The one or more non-transitory computer readable media of claim 1, wherein the operations further comprise:
identifying n highest similarity measures of the plurality of similarity measures; and
presenting standard codes, mapped to embedding vectors that correspond to the n highest similarity measures, as candidate standard codes for mapping to the target unmapped allergy code.

11. The one or more non-transitory computer readable media of claim 1, wherein the operations further comprise:
identifying a subset of similarity measures, of the plurality of similarity measures, that meet a threshold similarity measure; and
presenting standard codes, mapped to embedding vectors that correspond to the subset of similarity measures, as candidate standard codes for mapping to the target unmapped allergy code.

12. A method comprising:
accessing a plurality of standard codes, each standard code being mapped to a corresponding set of attributes;
generating a plurality of vector embeddings corresponding respectively to the plurality of standard codes, wherein generating the plurality of vector embeddings comprises:
applying a machine learning model to text of a first set of attributes associated with a first standard code of the plurality of standard codes for a first patient allergy event, to generate a first vector embedding;
accessing patient allergy data of a patient from one or more sources, wherein the patient allergy data comprises a target unmapped allergy code corresponding to a target allergy event, the target unmapped allergy code comprises allergy free text;
applying a machine learning model to the allergy free text of the target unmapped allergy code to generate a target vector embedding for the target unmapped allergy code;
computing a similarity measure for the target vector embedding and each of the plurality of vector embeddings to generate a plurality of similarity measures, the plurality of similarity measures comprise:
a first similarity measure for the target vector embedding and the first vector embedding; and
based at least on the first similarity measure, presenting the first standard code as a candidate standard code for mapping to the target unmapped allergy code,
wherein the method is performed by at least one device including a hardware processor.

13. The method of claim 12, wherein the plurality of standard codes comprise:
a. a first set of standard codes provided by a first entity, and
b. a second set of standard codes provided by a second entity, the first set of standard codes being different from the second set of standard codes, and the first entity being different from the second entity.

14. The method of claim 13, wherein the first set of standard codes comprises RxNorm codes and set of standard codes comprises SNOMED CT codes.

15. The method of claim 13, wherein a first candidate standard code from the first set of standard codes is prioritized over a candidate standard code from the second set of standard codes.

16. The method of claim 12, wherein generating the plurality of vector embeddings further comprises:
applying the machine learning model to text of a second set of attributes mapped to a second standard code, of the plurality of standard codes, for a second allergy event, to generate a second vector embedding;
wherein the plurality of similarity measures further comprise:
a second similarity measure for the target vector embedding and the second vector embedding;
the method further comprising:
based at least on the second similarity measure, refraining from presenting the second standard code as a candidate standard code for mapping to the target unmapped allergy code.

17. The method of claim 12, wherein the patient allergy data further comprises a second allergy event, wherein a second standard code corresponds to the second allergy event, wherein the method further comprises:
identifying that the second standard code associated with the second allergy event is the same as first standard code associated with the first allergy event; and
removing one of the target or second allergy event from the patient allergy data as duplicative of the other of the target or second allergy event.

18. The method of claim 12, further comprising,
identifying one or more standard codes that are similar to the first standard code;
generating a first grouping comprising the first standard code and the one or more similar standard codes,
wherein presenting the first standard code further comprises presenting the first grouping.

19. The method of claim 18, wherein the patient allergy data further comprises a second allergy event, wherein a second standard code corresponds to the second allergy event, wherein the method further comprises:
identifying that the second standard code associated with the second allergy event corresponds to one of the one or more similar standard codes in the first grouping; and
removing one of the target or second allergy event from the patient allergy data as duplicative of the other of the first or second allergy event.

20. A system comprising:
at least one device including a hardware processor;
the system being configured to perform operations comprising:
accessing a plurality of standard codes, each standard code being mapped to a corresponding set of attributes;
generating a plurality of vector embeddings corresponding respectively to the plurality of standard codes, wherein generating the plurality of vector embeddings comprises:
applying a machine learning model to text of a first set of attributes associated with a first standard code of the plurality of standard codes for a first patient allergy event, to generate a first vector embedding;
accessing patient allergy data of a patient from one or more sources, wherein the patient allergy data comprises a target unmapped allergy code corresponding to a target allergy event, the target unmapped allergy code comprises allergy free text;
applying a machine learning model to the allergy free text of the target unmapped allergy code to generate a target vector embedding for the target unmapped allergy code;
computing a similarity measure for the target vector embedding and each of the plurality of vector embeddings to generate a plurality of similarity measures, the plurality of similarity measures comprise:
a first similarity measure for the target vector embedding and the first vector embedding; and
based at least on the first similarity measure, presenting the first standard code as a candidate standard code for mapping to the target unmapped allergy code.
